SEQUENCE LISTING (156 sequences)

SEQ ID NO: 1 - DNA, 20 bp, Homo sapiens
actccaggctctgagacagc

SEQ ID NO: 2 - DNA, 20 bp, Homo sapiens
caccttgtcgaagtcctcct

SEQ ID NO: 3 - DNA, 20 bp, Homo sapiens
tattcgggaaagcctggtct

SEQ ID NO: 4 - DNA, 20 bp, Homo sapiens
cctcgcttttcctgaactct

SEQ ID NO: 5 - DNA, 20 bp, Homo sapiens
ggagcctgctggtcatttct

SEQ ID NO: 6 - DNA, 20 bp, Homo sapiens
caggaagggattctcaccag

SEQ ID NO: 7 - DNA, 20 bp, Homo sapiens
gccatttgagaattccctgt

SEQ ID NO: 8 - DNA, 20 bp, Homo sapiens
ggctttgcaattaccttcgt

SEQ ID NO: 9 - DNA, 20 bp, Homo sapiens
gcgtgctttatgaaggtggt

SEQ ID NO: 10 - DNA, 20 bp, Homo sapiens
ttctggatcgtggacaaaca

SEQ ID NO: 11 - DNA, 20 bp, Homo sapiens
gaggaaggcacttcacgttc

SEQ ID NO: 12 - DNA, 20 bp, Homo sapiens
actcccagactcgggaaatc

SEQ ID NO: 13 - DNA, 20 bp, Homo sapiens
cggcagtcaggacttaccat

SEQ ID NO: 14 - DNA, 20 bp, Homo sapiens
ccatgccagtgtcacctcta

SEQ ID NO: 15 - DNA, 20 bp, Homo sapiens
aggttgggatcattcagcat

SEQ ID NO: 16 - DNA, 20 bp, Homo sapiens
aacccagcaatctaggctct

SEQ ID NO: 17 - DNA, 20 bp, Homo sapiens
aaagcaggcattggtgatct

SEQ ID NO: 18 - DNA, 20 bp, Homo sapiens
agttccagaccaacctggtg

SEQ ID NO: 19 - DNA, 20 bp, Homo sapiens
gtttctcccccgtgtgaata

SEQ ID NO: 20 - DNA, 20 bp, Homo sapiens
atcgcaagtcattcctcacc

SEQ ID NO: 21 - DNA, 20 bp, Homo sapiens
ctgagatttccaacgcctgt

SEQ ID NO: 22 - DNA, 20 bp, Homo sapiens
ccccaggccatagtaccttt

SEQ ID NO: 23 - DNA, 20 bp, Homo sapiens
aaaacttcagaaggcggtga

SEQ ID NO: 24 - DNA, 22 bp, Homo sapiens
caccagtaaatcactgccaaaa

SEQ ID NO: 25 - DNA, 20 bp, Homo sapiens
acaccaccaagctccttcac

SEQ ID NO: 26 - DNA, 20 bp, Homo sapiens
ccgaggcattgtcatttctt

SEQ ID NO: 27 - DNA, 20 bp, Homo sapiens
atgtccctgaaacgaatgct

SEQ ID NO: 28 - DNA, 20 bp, Homo sapiens
atctcttgcccagcatcatc

SEQ ID NO: 29 - DNA, 20 bp, Homo sapiens
actgtgtgcgaatcatcagc

SEQ ID NO: 30 - DNA, 20 bp, Homo sapiens
cattgggaggtccaatgaag

SEQ ID NO: 31 - DNA, 20 bp, Homo sapiens
tggccactgcacatgtctat

SEQ ID NO: 32 - DNA, 20 bp, Homo sapiens
cttttccacagcatccttca

SEQ ID NO: 33 - DNA, 20 bp, Homo sapiens
ggaaaacaaccaggaatcca

SEQ ID NO: 34 - DNA, 19 bp, Homo sapiens
ttttgggcccagagaagac

SEQ ID NO: 35 - DNA, 20 bp, Homo sapiens
cattttgcagacgctctcag

SEQ ID NO: 36 - DNA, 20 bp, Homo sapiens
aacctccaccatgggaaaat

SEQ ID NO: 37 - DNA, 19 bp, Homo sapiens
gtctgacaaacgggagcag

SEQ ID NO: 38 - DNA, 17 bp, Homo sapiens
cccagtgctcggcacac

SEQ ID NO: 39 - DNA, 20 bp, Homo sapiens
acaccagcaaaggtgcctac

SEQ ID NO: 40 - DNA, 20 bp, Homo sapiens
ctccagatgcctggtgaaac

SEQ ID NO: 41 - DNA, 23 bp, Homo sapiens
tctgcttgtgtacctgactcatt

SEQ ID NO: 42 - DNA, 20 bp, Homo sapiens
ctgggaaattgccactctgt

SEQ ID NO: 43 - DNA, 25 bp, Homo sapiens
tttcaatgctattctttgtcatctt

SEQ ID NO: 44 - DNA, 20 bp, Homo sapiens
aagaaggctggagatggtca

SEQ ID NO: 45 - DNA, 20 bp, Homo sapiens
aagacaacgacgaccctcag

SEQ ID NO: 46 - DNA, 20 bp, Homo sapiens
tgcaagcgctgattaagaaa

SEQ ID NO: 47 - DNA, 20 bp, Homo sapiens
ttggcccttccatctgatac

SEQ ID NO: 48 - DNA, 20 bp, Homo sapiens
aaaggtaggtgccaccaatg

SEQ ID NO: 49 - DNA, 20 bp, Homo sapiens
cagagcagcctcccataatc

SEQ ID NO: 50 - DNA, 24 bp, Homo sapiens
tcaaaaatgagaggacatgtagga

SEQ ID NO: 51 - DNA, 17 bp, Homo sapiens
gctggggagacctggtg

SEQ ID NO: 52 - DNA, 21 bp, Homo sapiens
gagtaagtgatcctccccgac

SEQ ID NO: 53 - DNA, 20 bp, Homo sapiens
cctttcagcctcctcttcct

SEQ ID NO: 54 - DNA, 20 bp, Homo sapiens
tgtctttggcagacagaagg

SEQ ID NO: 55 - DNA, 20 bp, Homo sapiens
cggttggattcaacttaccc

SEQ ID NO: 56 - DNA, 23 bp, Homo sapiens
ggcagtagttttgttgctactga

SEQ ID NO: 57 - DNA, 21 bp, Homo sapiens
gggatagagggttttcagagc

SEQ ID NO: 58 - DNA, 20 bp, Homo sapiens
catttgctggggatttctgt

SEQ ID NO: 59 - DNA, 20 bp, Homo sapiens
cccacaactgtcagagcaaa

SEQ ID NO: 60 - DNA, 20 bp, Homo sapiens
tcctgaaggtgctcaaggtc

SEQ ID NO: 61 - DNA, 20 bp, Homo sapiens
ggagacaatgtggtccctga

SEQ ID NO: 62 - DNA, 21 bp, Homo sapiens
catgaatggcctcatcatctt

SEQ ID NO: 63 - DNA, 20 bp, Homo sapiens
gttccaccttgctgaagagc

SEQ ID NO: 64 - DNA, 20 bp, Homo sapiens
ccacaggtttgacttgcaga

SEQ ID NO: 65 - DNA, 20 bp, Homo sapiens
cttggtacccaagggcagta

SEQ ID NO: 66 - DNA, 20 bp, Homo sapiens
ctgcttgttgctgtcttcca

SEQ ID NO: 67 - DNA, 20 bp, Homo sapiens
attgagggccttcatctcct

SEQ ID NO: 68 - DNA, 20 bp, Homo sapiens
gctggcactatctccaaggt

SEQ ID NO: 69 - DNA, 20 bp, Homo sapiens
agcccaattctgaactgcat

SEQ ID NO: 70 - DNA, 18 bp, Homo sapiens
atcaagctggccctggac

SEQ ID NO: 71 - DNA, 20 bp, Homo sapiens
taggtggcaatctccatgtc

SEQ ID NO: 72 - DNA, 20 bp, Homo sapiens
agacaatgccctgaaggatg

SEQ ID NO: 73 - DNA, 20 bp, Homo sapiens
taggtggcaatctccatgtc

SEQ ID NO: 74 - DNA, 20 bp, Homo sapiens
agacaatgccctgaaggatg

SEQ ID NO: 75 - DNA, 19 bp, Homo sapiens
ctgaattcccccaggaaag

SEQ ID NO: 76 - DNA, 21 bp, Homo sapiens
tggcagaggagtaggtagtgg

SEQ ID NO: 77 - DNA, 24 bp, Homo sapiens
ctggattagtgcagatatttcaga

SEQ ID NO: 78 - DNA, 25 bp, Homo sapiens
actttctcttgctttctcaaacagg

SEQ ID NO: 79 - DNA, 21 bp, Homo sapiens
ggagggtactgtgctcacaaa

SEQ ID NO: 80 - DNA, 19 bp, Homo sapiens
ctctcccatcaccgtcctc

SEQ ID NO: 81 - DNA, 20 bp, Homo sapiens
agtgcctcatctgggtcttg

SEQ ID NO: 82 - DNA, 23 bp, Homo sapiens
tgaagacatctcaccttgaaaca

SEQ ID NO: 83 - DNA, 19 bp, Homo sapiens
gacgctcgatgtccaggtt

SEQ ID NO: 84 - DNA, 21 bp, Homo sapiens
gcaagaagtccaagctagaat

SEQ ID NO: 85 - DNA, 20 bp, Homo sapiens
tgcaggagtggtgaccataa

SEQ ID NO: 86 - DNA, 19 bp, Homo sapiens
tgacatcctctgcacttcc

SEQ ID NO: 87 - DNA, 20 bp, Homo sapiens
tggtattgcttggtgagcat

SEQ ID NO: 88 - DNA, 20 bp, Homo sapiens
caatggccgacattaccata

SEQ ID NO: 89 - DNA, 20 bp, Homo sapiens
tgctctttcagcgacatcac

SEQ ID NO: 90 - DNA, 20 bp, Homo sapiens
tttgtcgaggtcagaggtca

SEQ ID NO: 91 - DNA, 22 bp, Homo sapiens
catgtgttcacttctggtcgat

SEQ ID NO: 92 - DNA, 20 bp, Homo sapiens
caagggttttgggaatgatg

SEQ ID NO: 93 - DNA, 20 bp, Homo sapiens
atcgtcctcaccttcaccac

SEQ ID NO: 94 - DNA, 21 bp, Homo sapiens
tcctttaaagctgcaaagtcg

SEQ ID NO: 95 - DNA, 20 bp, Homo sapiens
ctccttgcccttctcatggt

SEQ ID NO: 96 - DNA, 20 bp, Homo sapiens
tgtgcctaccacgtaaccaa

SEQ ID NO: 97 - DNA, 20 bp, Homo sapiens
aggccaaaagtcccttctgt

SEQ ID NO: 98 - DNA, 20 bp, Homo sapiens
ggcctataagcctccctgag

SEQ ID NO: 99 - DNA, 20 bp, Homo sapiens
aatgctggacctggaggtag

SEQ ID NO: 100 - DNA, 20 bp, Homo sapiens
gggcgtgagagactgagaac

SEQ ID NO: 101 - DNA, 20 bp, Homo sapiens
tggagagccgagttcattct

SEQ ID NO: 102 - DNA, 20 bp, Homo sapiens
tccacaccaaagaggcaaag

SEQ ID NO: 103 - DNA, 18 bp, Homo sapiens
ggctgagtctcggctgaa

SEQ ID NO: 104 - DNA, 19 bp, Homo sapiens
accggagcctcgagttgac

SEQ ID NO: 105 - DNA, 20 bp, Homo sapiens
cagttccaatgggctgtctc

SEQ ID NO: 106 - DNA, 20 bp, Homo sapiens
agaggctgaagccactacca

SEQ ID NO: 107 - DNA, 20 bp, Homo sapiens
actttgggccgagaaaagat

SEQ ID NO: 108 - DNA, 20 bp, Homo sapiens
ctgctgcgtgagctggtact

SEQ ID NO: 109 - DNA, 20 bp, Homo sapiens
agctgcatacaggggtccag

SEQ ID NO: 110 - DNA, 20 bp, Homo sapiens
tcattcaggtctgcgatgag

SEQ ID NO: 111 - DNA, 20 bp, Homo sapiens
ctgagaaagccacggtgaca

SEQ ID NO: 112 - DNA, 20 bp, Homo sapiens
ccaaagttggccttcatgtt

SEQ ID NO: 113 - DNA, 20 bp, Homo sapiens
gagtgtgtggggtgtctgtg

SEQ ID NO: 114 - DNA, 20 bp, Homo sapiens
tgacgcccagaagtctatcc

SEQ ID NO: 115 - DNA, 19 bp, Homo sapiens
gctaccgatgggatagtcg

SEQ ID NO: 116 - DNA, 20 bp, Homo sapiens
cggaaatgtgcctgtttctt

SEQ ID NO: 117 - DNA, 19 bp, Homo sapiens
atagggggctggagtgaca

SEQ ID NO: 118 - DNA, 20 bp, Homo sapiens
catgaggctggcagtagtcc

SEQ ID NO: 119 - DNA, 18 bp, Homo sapiens
agttggcaggcaccacaa

SEQ ID NO: 120 - DNA, 20 bp, Homo sapiens
tttcagggtcatcccttcag

SEQ ID NO: 121 - DNA, 18 bp, Homo sapiens
caggagccccatgcattc

SEQ ID NO: 122 - DNA, 20 bp, Homo sapiens
gcccttccacaacatcacac

SEQ ID NO: 123 - DNA, 20 bp, Homo sapiens
acccagtagccaacacttgc

SEQ ID NO: 124 - DNA, 20 bp, Homo sapiens
tgaggatgacatcaccttgg

SEQ ID NO: 125 - DNA, 19 bp, Homo sapiens
gggagatcgaggcagattt

SEQ ID NO: 126 - DNA, 20 bp, Homo sapiens
cgcgtagggctttatcagtt

SEQ ID NO: 127 - DNA, 20 bp, Homo sapiens
tggtcttgaatgggatggtt

SEQ ID NO: 128 - DNA, 20 bp, Homo sapiens
gagcggaggatcgtgtactg

SEQ ID NO: 129 - DNA, 20 bp, Homo sapiens
tgggccttcatcacctctac

SEQ ID NO: 130 - DNA, 19 bp, Homo sapiens
agccagtgcccctctctaa

SEQ ID NO: 131 - DNA, 21 bp, Homo sapiens
agttacccaagagtgccaaga

SEQ ID NO: 132 - DNA, 20 bp, Homo sapiens
tgcccacctcttcctttaca

SEQ ID NO: 133 - DNA, 20 bp, Homo sapiens
ggaacagtcatcgttgtcca

SEQ ID NO: 134 - DNA, 20 bp, Homo sapiens
catggaattgtcacccagaa

SEQ ID NO: 135 - DNA, 20 bp, Homo sapiens
agcaccagtcatgcaggttt

SEQ ID NO: 136 - DNA, 20 bp, Homo sapiens
cctttggctgtgtcaggcta

SEQ ID NO: 137 - DNA, 21 bp, Homo sapiens
cacctgacttctgatgtgcaa

SEQ ID NO: 138 - DNA, 22 bp, Homo sapiens
cccattactacctgcactttgg

SEQ ID NO: 139 - DNA, 21 bp, Homo sapiens
aggtgtccacagagctggtaa

SEQ ID NO: 140 - DNA, 20 bp, Homo sapiens
tcgatgtgctagtgctttgg

SEQ ID NO: 141 - DNA, 18 bp, Homo sapiens
gggtctccaccgatgaca

SEQ ID NO: 142 - DNA, 19 bp, Homo sapiens
gggggtgtagaggccaaag

SEQ ID NO: 143 - DNA, 18 bp, Homo sapiens
cggctcctgtggctcaga

SEQ ID NO: 144 - DNA, 19 bp, Homo sapiens
ctggagctggaagttgctg

SEQ ID NO: 145 - DNA, 20 bp, Homo sapiens
gccttgtcagctggagattg

SEQ ID NO: 146 - DNA, 20 bp, Homo sapiens
accagctcagaccatgtgga

SEQ ID NO: 147 - DNA, 20 bp, Homo sapiens
ccatcttctccgtgatttcc

SEQ ID NO: 148 - DNA, 20 bp, Homo sapiens
cgaccgggtttatgaaattg

SEQ ID NO: 149 - DNA, 26 bp, Homo sapiens
aaaaatttgttttcaccttactttca

SEQ ID NO: 150 - DNA, 22 bp, Homo sapiens
caagtgaacctgaaatgattgg

SEQ ID NO: 151 - DNA, 19 bp, Homo sapiens
cagcttcatggctgaagga

SEQ ID NO: 152 - DNA, 20 bp, Homo sapiens
acaccctgaggagaatcacg

SEQ ID NO: 153 - DNA, 18 bp, Homo sapiens
atggcccagatgctgacc

SEQ ID NO: 154 - DNA, 20 bp, Homo sapiens
ccctcaaagacactccaacc

SEQ ID NO: 155 - DNA, 18 bp, Homo sapiens
gggatcatgtccctcacg

SEQ ID NO: 156 - DNA, 20 bp, Homo sapiens
cctccaagcagctgaagagt

(19) United States
(12) Patent Application Publication
(10) Pub. No.: US 2015/0259741 A1
(43) Pub. Date: Sep. 17, 2015

(54) METHODS AND COMPOSITIONS FOR THE DIAGNOSIS OF MULTIPLE SCLEROSIS

(71) Applicants: LINEAGEN, INC., Salt Lake City, UT (US); THE UNIVERSITY OF UTAH RESEARCH FOUNDATION, Salt Lake City, UT (US)

(72) Inventors: Mark F. Leppert, Salt Lake City, UT (US); John W. Rose, Salt Lake City, UT (US); Nori Matsunami, Salt Lake City, UT (US); Charles Henry Hensel, Salt Lake City, UT (US)

(21) Appl. No.: 14/441,081

(22) PCT Filed: Nov. 6, 2013
(86) PCT No.: PCT/US13/68765

§ 371 Date: May 6, 2015

Related U.S. Application Data
(60) Provisional application No. 61/723,077, filed on Nov. 6, 2012.

(51) Int. Cl. C12Q 1/68; C40B 30/02

(52) U.S. Cl. CPC C12Q 1/6883; C40B 30/02; C12Q 2600/156; C12Q 2600/158

(57) ABSTRACT

Disclosed herein are methods and compositions for diagnosing multiple sclerosis (“MS”) in a subject or the risk of MS in a subject. More particularly, methods and compositions for the use of genetic markers for diagnosing MS in subject or the risk of MS in a subject.

### RELATED APPLICATIONS

This application claims priority to U.S. Provisional Application No. 61/723,077, entitled “Haplotype Sharing and Linkage Analyses of Multigenerational Families with Multiple Sclerosis” and filed on Nov. 6, 2012, which is incorporated herein by reference in its entirety.

### TECHNICAL FIELD

The present disclosure relates to methods and compositions for determining the risk of multiple sclerosis (MS) or the diagnosis of MS. The present disclosure also relates to the use of genetic markers for determining risk of MS and the diagnosis of MS.

### BACKGROUND

MS is an autoimmune disease that affects the central nervous system (CNS). The CNS consists of the brain, spinal cord, and the optic nerves. Surrounding and protecting the nerve fibers of the CNS is a fatty tissue called myelin which helps nerve fibers conduct electrical impulses. In MS, myelin is lost in multiple areas, leaving scar tissue called sclerosis. These damaged areas are also known as plaques or lesions. Sometimes the nerve fiber itself is damaged or broken. When myelin or the nerve fiber is destroyed or damaged, the ability of the nerves to conduct electrical impulses to and from the brain is disrupted, and this produces the various symptoms of MS.

MS is a complex disease with heterogeneous disease course, neuropathology and gender bias. The disorder features autoimmunity, inflammation, neurodegeneration and impaired regeneration. Distinct neuropathologies are now being associated with the progressive and relapsing states of the disease. In terms of etiology, family studies have shown that MS has a genetic component. Additionally, there are likely a number of environmental factors, such as exposure to certain pathogens or damage mechanisms, which might increase MS susceptibility.

People with MS can expect one of four clinical courses of disease, each of which might be mild, moderate, or severe. These include Relapsing-Remitting (RR), Primary-Progressive (PP), Secondary-Progressive (SP), and Progressive-Relapsing (PR). Individuals with RR MS experience clearly defined flare-ups (also called relapses, attacks, or exacerbations). These are episodes of acute worsening of neurologic function. They are followed by partial or complete recovery periods (remissions) free of disease progression. Individuals with PP MS experience a slow but nearly continuous worsening of their disease from the onset, with no distinct relapses or remissions. However, there are variations in rates of progression over time, occasional plateaus, and temporary minor improvements. Individuals with SP MS experience an initial period of relapsing-remitting disease, followed by a steadily worsening disease course with or without occasional flare-ups, minor recoveries (remissions), or plateaus. Individuals with PR MS experience a steadily worsening disease from the onset but also have clear acute relapses (attacks or exacerbations), with or without recovery. In contrast to RR MS, the periods between relapses are characterized by continuing disease progression.

Patients can progress rapidly over several months to death, or may have a few relapses and then remain clinically stable for many decades. It is difficult to predict which patients will progress and which will remain relatively stable. Although there are clearly patients in whom the disease remains benign, it is very difficult to predict which course a patient's disease will follow.

At this time, there is no cure for MS. Despite treatment with available agents, a majority of patients eventually progress to a SP stage of disease leading to severe disability. The ability to identify individuals who have a risk of MS and to reliably diagnose MS would be very valuable to increase the likelihood of successful treatment.

### BRIEF DESCRIPTION OF THE DRAWINGS

FIG. 1 shows a multigenerational MS pedigree. Affy 6.0 indicates samples genotyped with an Affymetrix Genome-Wide Human SNP array 6.0. The circled samples were used for phased haplotype sharing analysis. The arrows point to samples used for custom targeted enrichment and next-gen DNA sequencing.

FIGS. 2A and 2B show the results of a phased haplotype sharing analysis.

FIG. 3 shows the overlap of the Utah K1601 chromosome 12 MS region 12p12.3-q12 with a MS region described in another multiplex MS family.

### DETAILED DESCRIPTION

Disclosed are molecules, materials, compositions, and components that can be used for, can be used in conjunction with, can be used in preparation for, or are products of the disclosed methods and compositions. These and other materials are disclosed herein, and it is understood that when combinations, subsets, interactions, groups, etc., of these materials are disclosed that while specific reference of each various individual and collective combinations and permutation of these molecules and compounds may not be explicitly disclosed, each is specifically contemplated and described herein. For example, if a nucleotide or nucleic acid is disclosed and discussed and a number of modifications that can be made to a number of molecules including the nucleotide or nucleic acid are discussed, each and every combination and permutation of nucleotide or nucleic acid and the modifications that are possible are specifically contemplated unless specifically indicated to the contrary. This concept applies to all aspects of this application including, but not limited to, steps in methods of making and using the disclosed molecules and compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the meanings that would be commonly understood by one of skill in the art in the context of the present specification.

It must be noted that as used herein and in the appended claims, the singular forms “a,” “an,” and “the” include plural reference unless the context clearly dictates otherwise. Thus, for example, reference to “a nucleotide” includes a plurality of such nucleotides; reference to “the nucleotide” is a reference to one or more nucleotides and equivalents thereof known to those skilled in the art, and so forth.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. Unless otherwise specified, the term “patient” includes human and veterinary subjects.

As used herein, the term “biomarker” or “biological marker” means an indicator of a biologic state and may include a characteristic that is objectively measured as an indicator of normal biological processes, pathologic processes, or pharmacologic responses to a therapeutic or other intervention. In one embodiment, a biomarker may indicate a change in expression or state of a protein that correlates with the risk or progression of a disease, or with the susceptibility of the disease in an individual. In certain embodiments, a biomarker may include one or more of the following: genes, proteins, glycoproteins, metabolites, cytokines, and antibodies.

As used herein, the term “in vitro diagnostic” means diagnostic tests that may be used to detect or indicate the presence of, the predisposition to, or the risk of, diseases, conditions, infections and/or therapeutic responses. In one embodiment, an in vitro diagnostic may be used in a laboratory or other health professional setting. In another embodiment, an in vitro diagnostic may be used by a consumer at home. In vitro diagnostic products are those reagents, instruments, and systems intended for use in the in vitro diagnosis of disease or other conditions, including a determination of the state of health, in order to cure, mitigate, treat, or prevent disease or its sequelae. In one embodiment, in vitro diagnostic products may be intended for use in the collection, preparation, and examination of specimens taken from the human body. In certain embodiments, in vitro diagnostic products may comprise one or more laboratory tests such as one or more in vitro diagnostic tests. As used herein, the term “laboratory test” means one or more medical or laboratory procedures that involve testing samples of blood, urine, or other tissues or substances in the body.

In one embodiment, the methods and in vitro diagnostic products described herein may be used for the diagnosis of MS in at-risk patients, patients with non-specific symptoms possibly associated with MS, and/or patients presenting with Clinically Isolated Syndrome. In another embodiment, the methods and in vitro diagnostic products described herein may be used for screening for risk of progressing from at-risk, non-specific symptoms possibly associated with MS, and/or Clinically Isolated Syndrome to fully-diagnosed MS. In certain embodiments, the methods and in vitro diagnostic products described herein can be used to rule out screening of diseases and disorders that share symptoms with MS. In yet another embodiment, the methods and in vitro diagnostic products described herein may indicate diagnostic information to be included in the current diagnostic evaluation in patients suspected of having MS.

A drug or pharmaceutical agent means any substance used in the prevention, diagnosis, alleviation, treatment or cure of a disease. These terms include a vaccine, for example.

The present disclosure also includes nucleic acid molecules that are oligonucleotides capable of hybridizing, under stringent hybridization conditions, with complementary regions of a gene or chromosome region containing a polymorphism or variant allele of the present disclosure. A nucleic acid can be DNA or RNA, and single- or double-stranded. Oligonucleotides can be naturally occurring or synthetic, but are typically prepared by synthetic means. Preferred oligonucleotides of the invention include segments of DNA, or their complements. The segments are usually between 5 and 200 contiguous bases, and often range from 5, 10, 12, 15, 20, or 25 nucleotides to 10, 15, 30, 25, 20, 50, 100, 150 or 200 nucleotides. Nucleic acids between 5-10, 5-20, 10-20, 12-30, 15-30, 10-50, 20-50, 20-100, or 20-200 bases are common. The variant allele or polymorphic site can occur within any position of the segment of DNA, gene, or chromosome region.

Oligonucleotides of the present disclosure can be RNA, DNA, or derivatives of either. The minimum size of such oligonucleotides is the size required for formation of a stable hybrid between an oligonucleotide and a complementary sequence on a nucleic acid molecule of the present disclosure. The present disclosure includes oligonucleotides that can be used as, for example, probes to identify nucleic acid molecules or primers to produce nucleic acid molecules. Oligonucleotide probes or primers may include a single base change of a variant or polymorphism of the present disclosure or the wildtype nucleotide that is located at the same position. In certain embodiments, the nucleotide of interest may occupy a central position of a probe. In one embodiment, the nucleotide of interest occupies a 3′ position of a primer.

In another embodiment of the present disclosure, an array of oligonucleotides are provided, where discrete positions on the array are complementary to one or more of the variants disclosed herein. Such an array may comprise a series of oligonucleotides, each of which can specifically hybridize to particular nucleotide variant or polymorphism. Arrays of interest may further comprise sequences, including polymorphisms, of other genetic sequences, particularly other sequences of interest for pharmacogenetic screening. As with other human polymorphisms, the polymorphisms and variants of the disclosure also have more general applications, such as forensic, paternity testing, linkage analysis and positional cloning.

Described herein are methods directed to identifying subjects predisposed to MS or with a risk of developing MS. Also described herein are methods for diagnosing MS in a subject. In one embodiment, the methods disclosed may be used to characterize the clinical course or status of MS in a subject. In one embodiment, the methods as disclosed herein may be used to predict a response in a subject to an existing treatment for MS, or a treatment for MS that is in development or has yet to be developed. In one embodiment, the methods may be used to determine whether a patient may be more or less responsive to immunotherapies. In another embodiment, the methods described herein may be used to predict a response to a treatment with one or more immunological agents. In another embodiment, the methods may be used to predict a response to a treatment with Copaxone®. In another embodiment, the methods described herein may be used to predict the response to a therapy with Tysabri®.

In one embodiment, the presence or absence of certain genetic markers, such as one or more variant alleles, may be used to identify individuals that may have MS, are predisposed to MS, or have a risk or susceptibility to developing MS. As used herein, the term “susceptibility” or “susceptible” means that an individual has MS or is predisposed or at risk of developing MS.

In yet another embodiment, the variant alleles disclosed herein may be used for the stratification of MS patients according to their disease status, progression or the predicted response to one or more MS therapies. In another embodiment, one or more clinical, neuroradiological, genetic and/or immunological markers may be used to predict the response of a subject to one or more treatments or therapies for MS. In one such embodiment, the presence or absence of certain genetic markers, such as variant alleles, may be used to predict the response to one or more MS therapies. In another embodiment, the presence or absence of certain phenotypic variables, along with certain variant alleles, may be used to diagnose MS in a subject. In yet another embodiment, the presence or absence of phenotypic markers and/or variant alleles may be used to determine the clinical status of a MS patient and whether a patient is more likely to have a favorable clinical outcome with a certain MS therapy.

In one embodiment, the presence or absence of one or more variant alleles may be used to indicate the clinical disease status of a subject. In one such embodiment, the presence or absence of one or more variant alleles may indicate whether a subject may be stratified or characterized as having one of four clinical courses of disease consisting of Relapsing-Remitting (RR), Primary-Progressive (PP), Secondary-Progressive (SP), and Progressive-Relapsing (PR).

The teachings disclosed herein provide a collection of functionally relevant MS variant alleles and polymorphisms in genes or chromosomal regions. Detection of polymorphisms is useful in designing and performing diagnostic assays for evaluation of genetic risks or susceptibility for MS and other related conditions. Analysis of polymorphisms is also useful in designing prophylactic and therapeutic regimes customized to MS treatments. Detection of polymorphisms is also useful for conducting clinical trials of drugs for treatment of MS.

Polymorphism refers to the occurrence of two or more genetically determined alternative nucleotide sequences or alleles in a population. A polymorphic genetic marker or site is the locus at which divergence occurs. In one embodiment, genetic markers have at least two alleles, each occurring at a frequency of greater than 1%, and more preferably greater than 10% or 20% of a selected population. A polymorphic locus may be as small as one base pair.

Polymorphic genetic markers may include single nucleotide polymorphisms (SNP), single nucleotide variants (SNV), restriction fragment length polymorphisms (RFLP), exonic variants, splicing variants, variant alleles, variable number of tandem repeats (VNTRs), hypervariable regions, minisatellites, dinucleotide repeats, trinucleotide repeats, tetranucleotide repeats, simple sequence repeats, and insertion elements.

A single nucleotide polymorphism (SNP) occurs at a polymorphic site occupied by a single nucleotide, which is the site of variation between allelic sequences. A SNP may arise due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

As used herein, the nucleotide sequences disclosed herein encompass the complements of said nucleotide sequences. In addition, as used herein, the term “SNP” encompasses any allele among a set of alleles. The term “allele” refers to a specific nucleotide among a selection of nucleotides defining a SNP. In certain embodiments, the alleles at the site of an SNP may be a reference allele or a variant allele.

In one embodiment, the presence or absence of one or more variant alleles, genetic markers, polymorphisms, or genetic variants may be predictive of whether an individual is at risk or susceptibly to MS. In one such embodiment, one or more genetic markers may be identified as being associated with a disease phenotype by the use of a genome wide association study (GWAS). As generally know by those of skill in the art, a GWAS is an examination of genetic polymorphism across a genome, designed to identify genetic associations with a trait or phenotype of interest, such as MS. If genetic polymorphisms are more frequent in people with MS, the variations are said to be “associated” with MS. The polymorphisms associated with MS may either directly cause the disease phenotype or they may be in linkage disequilibrium with nearby genetic mutations that influence the individual variation in the disease phenotype. Linkage disequilibrium, as used herein, is the non-random association of alleles at two or more loci. In certain embodiments, a GWAS may be accompanied by a phased haplotype sharing analysis.

In one embodiment, a GWAS may be conducted using a DNA microarray as generally known in the art. Array-based detection can be performed to detect genetic polymorphisms. Commercially available arrays, e.g. Affymetrix Genome-Wide Human SNP array 6.0, from Affymetrix, Inc. (Santa Clara, Calif.) or other manufacturers may be used to detect polymorphisms. Reviews regarding the operation of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11:A879; Fodor (1997) “Massively Parallel Genomics.” Science 277: 393-395; and Ghee et al. (1996) “Accessing Genetic Information with High-Density DNA Arrays.” Science 274:610-614, each of which is incorporated herein by reference.

As generally known in the art, a variety of probe arrays can be used for detection of polymorphisms that can be correlated to the phenotypes of interest. In one embodiment, DNA probe array chips or larger DNA probe array wafers (from which individual chips would otherwise be obtained by breaking up the wafer) may be used. In one such embodiment, DNA probe array wafers may comprise glass wafers on which high density arrays of DNA probes (short segments of DNA) have been placed. Each of these wafers can hold, for example, millions of DNA probes that are used to recognize sample DNA sequences (e.g., from individuals or populations that may comprise polymorphisms of interest). In certain embodiments, the DNA samples may be from individuals from multigenerational families with members that are affected and unaffected with MS. The recognition of sample DNA by the set of DNA probes on the glass wafer takes place through DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of nucleic acid is present or not in the sample, thereby determining whether a polymorphism found in the nucleic acid is present.

In one embodiment, the use of DNA probe arrays to obtain allele information typically involves the following general steps: design and manufacture of DNA probe arrays, preparation of the sample, hybridization of sample DNA to the array, detection of hybridization events and data analysis to determine the presence or absence of variant alleles. In one such embodiment, wafers may be manufactured using a process adapted from semiconductor manufacturing to achieve cost effectiveness and high quality, and are available, e.g., from Affymetrix, Inc. of Santa Clara, Calif.

In one embodiment, genetic markers used to diagnose MS, a predisposition or increased risk or susceptibility to MS, or a response to a MS therapeutic, may include one or more SNPs. As disclosed herein, a SNP may be identified by its name or by location within a particular sequence. The nucleotides flanking an SNP are the flanking sequences which may be used to identify the location of the SNP in the genome. In other embodiments, genetic markers used to diagnose MS, a predisposition or increased risk or susceptibility to MS, or a response to a MS therapeutic, may include one or more variant alleles.

In one embodiment, the variant alleles used to diagnose a predisposition or increased risk of MS, diagnose MS, or a response to a MS therapeutic, may include one or more loci located in a particular region of a chromosome. In one embodiment, the variant alleles may be located in a region of a chromosome selected from one or more of the chromosomal regions comprising 12p12.3-q12 and 16q21-q22.3. In another embodiment, the polymorphisms and variants used to diagnose MS, or a predisposition or increased risk of MS, may be one or more variants of one or more of the genes comprising C1orf125, PLD5, NCKAP5, NCKAP5, SCN9A, TUBA4A, ZNF717, NPHP3, LEKR1, EHHADH, AIF1, HIVEP2, RELN, IL2RA, CD6, RAB38, PTPRO, STRAP, PIK3C2G, PLEKHA5, PDE3A, GYS2, ERGIC2, ABCD2, COL2A1, OR10AD1, FMNL3, SLC11A2, KRT80, KRT75, KRT74, KRT76, KRT3, ITGB7, UTP20, TUBA3C, SLITRK6, NUBPL, SNX29, CNOT1, GOT2, CDH11, CDH16, C16orf70, ELMO3, FAM65A, RLTPR, PARD6A, C16orf48, TSNAXIP1, TSNAXIP1, SLC12A4, COG8, FUK, IL34, HYDIN, MARVELD3, PHLPP2, PKD1L3, ZFHX3, MLKL, FA2H, WDR59, ZNRF1, BCAR1, ADAT1, KARS, KIAA1012, and CPAMD8. In particular embodiments, the polymorphisms and variants used to diagnose MS, or a predisposition or increased risk of MS, may be one or more variant alleles described in Table 1 and Table 2.

The variant alleles as provided herein may include one or more variant alleles described in Table 1 and Table 2. The presence of variant alleles in a genetic sample may be determined by using one or more synthetic PCR primer sequences selected from the sequences identified by SEQ ID NOS: 1-156. In particular embodiments, the variant alleles of Table 1 may be identified using the forward and reverse primers sequences selected from SEQ ID NOS: 1-6. In one embodiment, the presence of the chromosome 16 variant allele of Table 1, in the gene ELMO3, at position chr16:67236368, may be assayed using the forward primer ACTCCAGGCTCTGAGACAGC (SEQ ID NO: 1) and the reverse primer CACCTTGTCGAAGTCCTCCT (SEQ ID NO: 2), wherein the variant allele is “A”. In another embodiment, the presence of the chromosome 16 variant allele of Table 1, in the gene ZFHX3 (ATBF1), at position chr16:72993489, may be assayed using the forward primer TATTCGGGAAAGCCTGGTCT (SEQ ID NO: 3) and the reverse primer CCTCGCTTTTCCTGAACTCT (SEQ ID NO: 4), wherein the variant allele is “C”. In a further embodiment, the presence of the chromosome 16 variant allele of Table 1, in the gene IL34, at position chr16:70690511, may be assayed using the forward primer GGAGCCTGCTGGTCATTTCT (SEQ ID NO: 5) and the reverse primer CAGGAAGGGATTCTCACCAG (SEQ ID NO: 6), wherein the variant allele is “C”.

In one embodiment, the methods disclosed herein may comprise assaying for the presence of one or more variant alleles or polymorphisms in an individual which may include methods generally known in the art. In one such embodiment, methods for assaying for the presence of one or more variant alleles in an individual may include assaying an individual for the presence or absence of one or more variant alleles using one or more genotyping assays such as a PCR assay, SNP array, PCR-based SNP genotyping, DNA hybridization, fluorescence microscopy, immunoassay, and other methods known by those of skill in the art. In another embodiment, methods for assaying the presence or absence of one or more SNP markers may include providing a nucleotide sample from an individual and assaying the nucleotide sample for the presence or absence of one or more SNP markers. In one such embodiment, the nucleotide sample may include, e.g., a biological fluid or tissue. Examples of biological fluids include, e.g., whole blood, serum, plasma, cerebrospinal fluid, urine, tears or saliva. Examples of tissue include, e.g., connective tissue, muscle tissue, nervous tissue, epithelial tissue, and combinations thereof.

In one embodiment, methods for diagnosing subjects with MS or individuals predisposed or at risk of developing MS are provided. In another embodiment, methods for predicting the response to a MS treatment or therapy are provided. In one embodiment, the method comprises the steps of obtaining a sample from a subject and assaying the sample for the presence of one or more variant alleles, polymorphisms, or genetic markers, wherein the presence of one or more variant alleles, polymorphisms, or genetic markers indicates subjects with MS or individuals predisposed or at risk of developing MS. In particular embodiments, the method comprises the steps of obtaining a sample from a subject and assaying the sample for the presence of one or more variant alleles selected from at least one variant allele listed in Table 1 and/or Table 2, wherein the presence of the one or more variant alleles listed in Table 1 and/or Table 2 indicates a subject with MS or predisposed or at risk of developing MS. In certain embodiments, the method comprises the steps of obtaining a sample from a subject and assaying the sample for the presence of at least one variant allele listed in Table 1, wherein the presence of the one or more variant alleles listed in Table 1 indicates a subject with MS or predisposed or at risk of developing MS. In one such embodiment, the sample is assayed for the presence of at least one of the variant alleles of Table 1 with a PCR assay using one or more of the forward and reverse primers sequences selected from SEQ ID NOS: 1-6. In another such embodiment, the sample is assayed for the presence of at least one of the variant alleles of Table 1 by assaying the sample for the presence of the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is “A”. In yet another such embodiment, the sample is assayed for the presence of at least one of the variant alleles of Table 1 by assaying the sample for the presence of the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is “C”. In still yet another such embodiment, the sample is assayed for the presence of at least one of the variant alleles of Table 1 by assaying the sample for the presence of the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is “C”. In other embodiments, the sample is assayed for the presence of at least one of the variant alleles of Table 1 by assaying the sample for the presence of the chromosome 16 variant allele of Table 1, in the gene ELMO3, at position chr16:67236368, in combination with one or both of the chromosome 16 variant allele of Table 1, in the gene ZFHX3 (ATBF1), at position chr16:72993489, and the chromosome 16 variant allele of Table 1, in the gene IL34, at position chr16:70690511.

In further embodiments, the method comprises the steps of obtaining a sample from a subject and assaying the sample for the presence of at least one variant allele listed in Table 2, wherein the presence of the one or more variant alleles listed in Table 2 indicates a subject with MS or predisposed or at risk of developing MS. In such embodiments, the sample may be assayed for the presence of at least one of the variant alleles of Table 2 using the forward and reverse primers sequences selected from SEQ ID NOS: 7-156.

Also disclosed herein are in vitro diagnostic products for detecting the risk on MS in a subject or diagnosis MS in a subject. The in vitro diagnostic products comprise at least one laboratory test for assaying a sample from a subject for the presence of one or more variant alleles, polymorphisms, or genetic markers, wherein the presence of one or more variant alleles, polymorphisms, or genetic markers indicates subjects with MS or individuals predisposed or at risk of developing MS. In particular embodiments, the laboratory test comprises the steps of obtaining a sample from a subject and assaying the sample for the presence of one or more variant alleles selected from at least one variant allele listed in Table 1 and/or Table 2, wherein the presence of the one or more variant alleles listed in Table 1 and/or Table 2 indicates a subject with MS or predisposed or at risk of developing MS. In certain embodiments, the laboratory test comprises the steps of obtaining a sample from a subject and assaying the sample for the presence of at least one variant allele listed in Table 1, wherein the presence of the one or more variant alleles listed in Table 1 indicates a subject with MS or predisposed or at risk of developing MS. In one such embodiment, the sample is assayed for the presence of at least one of the variant alleles of Table 1 with a PCR assay using one or more of the forward and reverse primers sequences selected from SEQ ID NOS: 1-6. In another such embodiment, the sample is assayed for the presence of at least one of the variant alleles of Table 1 by assaying the sample for the presence of the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is “A”. In yet another such embodiment, the sample is assayed for the presence of at least one of the variant alleles of Table 1 by assaying the sample for the presence of the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is “C”. In still yet another such embodiment, the sample is assayed for the presence of at least one of the variant alleles of Table 1 by assaying the sample for the presence of the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is “C”. In other embodiments, the sample is assayed for the presence of at least one of the variant alleles of Table 1 by assaying the sample for the presence of the chromosome 16 variant allele of Table 1, in the gene ELMO3, at position chr16:67236368, in combination with one or both of the chromosome 16 variant allele of Table 1, in the gene ZFHX3 (ATBF1), at position chr16:72993489, and the chromosome 16 variant allele of Table 1, in the gene IL34, at position chr16:70690511.

The following examples are given to illustrate various embodiments which have been made with the present invention. It is to be understood that the following examples are provided by way of illustration and nothing therein should be taken as a limitation upon the overall scope of the many embodiments which can be prepared in accordance with the present invention.

### EXAMPLES

The Examples that follow are offered for illustrative purposes only and are not intended to limit the scope of the compositions and methods described herein in any way. In each instance, unless otherwise specified, standard materials and methods were used in carrying out the work described in the Examples provided. All patent and literature references cited in the present specification are hereby incorporated by reference in their entirety.

The practice of the present invention employs, unless otherwise indicated, conventional techniques of chemistry, molecular biology, microbiology, recombinant DNA, genetics, immunology, cell biology, cell culture and transgenic biology, which are within the skill of the art (See, e.g., Maniatis, T., et al. (1982) Molecular Cloning: A Laboratory Manual (Cold Spring Harbor Laboratory, Cold Spring Harbor, N.Y.); Sambrook, J., et al. (1989) Molecular Cloning: A Laboratory Manual, 2^{nd} Ed. (Cold Spring Harbor Laboratory, Cold Spring Harbor, N.Y.); Ausubel, F. M., et al. (1992) Current Protocols in Molecular Biology, (J. Wiley and Sons, NY); Glover, D. (1985) DNA Cloning, I and II (Oxford Press); Anand, R. (1992) Techniques for the Analysis of Complex Genomes, (Academic Press); Guthrie, G. and Fink, G. R. (1991) Guide to Yeast Genetics and Molecular Biology (Academic Press); Harlow and Lane (1988) Antibodies: A Laboratory Manual (Cold Spring Harbor Laboratory, Cold Spring Harbor, N.Y.); Jakoby, W. B. and Pastan, I. H. (eds.) (1979) Cell Culture. Methods in Enzymology, Vol. 58 (Academic Press, Inc., Harcourt Brace Jovanovich (NY); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Hogan et al. (eds) (1994) Manipulating the Mouse Embryo. A Laboratory Manual, 2^{nd} Edition, Cold Spring Harbor Laboratory Press, Cold Spring Harbor, N.Y. A general discussion of techniques and materials for human gene mapping, including mapping of human chromosome 1, is provided, e.g., in White and Lalouel (1988) Ann. Rev. Genet. 22:259 279. The practice of the present invention employs, unless otherwise indicated, conventional techniques of chemistry, molecular biology, microbiology, recombinant DNA, genetics, and immunology. (See, e.g., Maniatis et al., 1982; Sambrook et al., 1989; Ausubel et al., 1992; Glover, 1985; Anand, 1992; Guthrie and Fink, 1991).

Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### Example 1

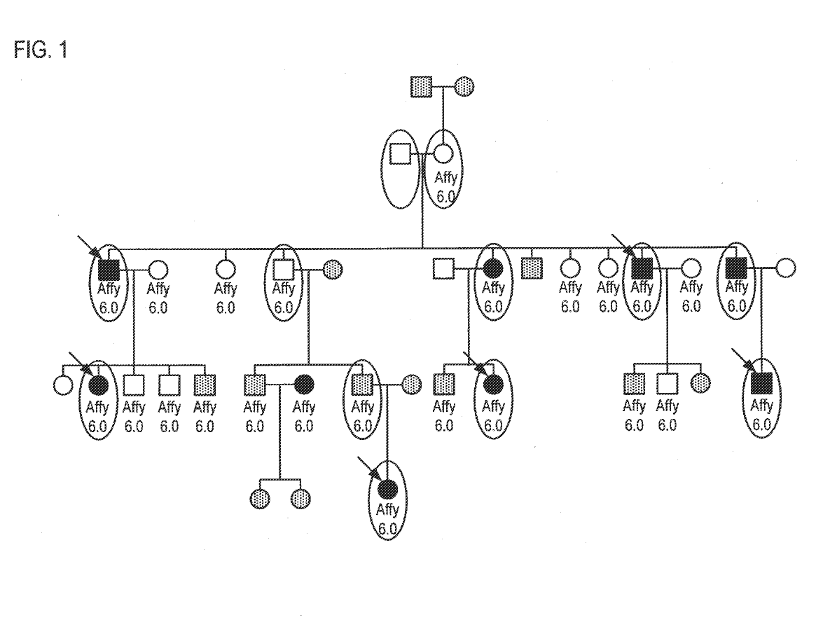

Genotyping experiments were performed on affected and unaffected members of 19 multigenerational Utah families with MS. Biologic samples were collected from members the 19 multigenerational Utah families with MS and selected samples were genotyped using the Affymetrix Genome-Wide Human SNP array 6.0. FIG. 1 shows the MS pedigree chart of Utah multigenerational family K1601. With continued reference to FIG. 1, the samples from the members of family K1601 labeled with “Affy 6.0” were selected for genotyping; the circled members indicate those samples used for phased haplotype sharing analysis (Example 2); and the arrows indicate the samples used from custom targeted enrichment and next-generation DNA sequencing (Example 4).

### Example 2

Phased haplotype sharing analysis was carried out on the Affymetrix Genome-Wide Human SNP array 6.0 genotype data using the HapShare method described in Arrington et al. (Arrington C B, et al., Am J Med Genet Part A 158A:3137-3147), the entirety of which is incorporated herein by reference. Briefly, the HapShare method examined sharing of phased haplotype data and was carried out in two steps. First, each phased paternal and maternal haplotype from the selected members of the 19 multigenerational Utah families with MS were compared to each other in a pair-wise manner to identify identical shared genomic segments. Shared segments were defined as regions where at least one informative inclusion marker (haplotype 1/haplotype 2=A/A or B/B) was flanked by exclusion markers (haplotype 1/haplotype 2=A/B or B/A) at both ends. Shared segments were not disrupted by non-informative calls. Next, when sharing was observed among family members with MS, that shared haplotype was compared against paternal and maternal haplotypes of members without MS to determine whether a shared genomic segment might be a common haplotype.

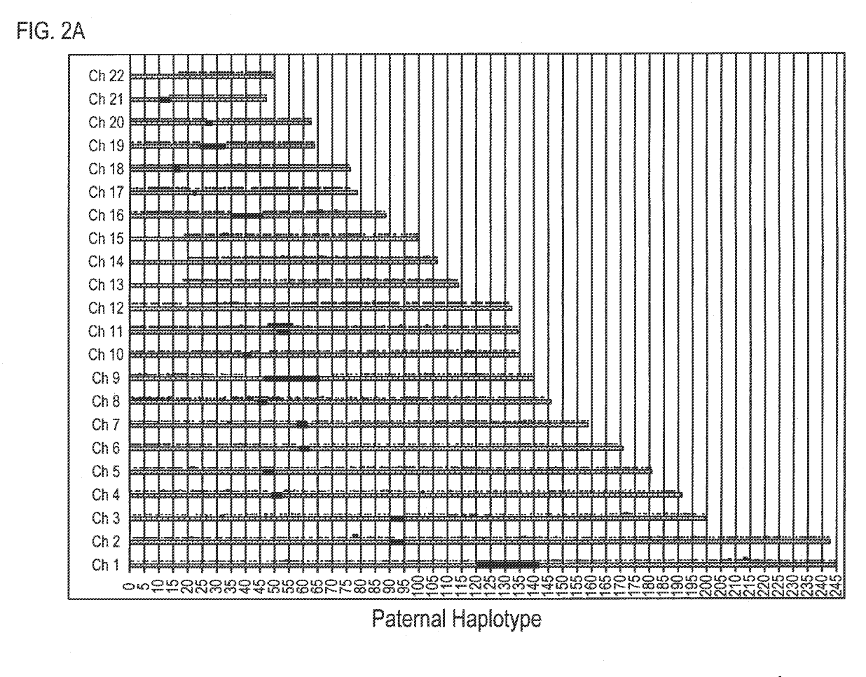

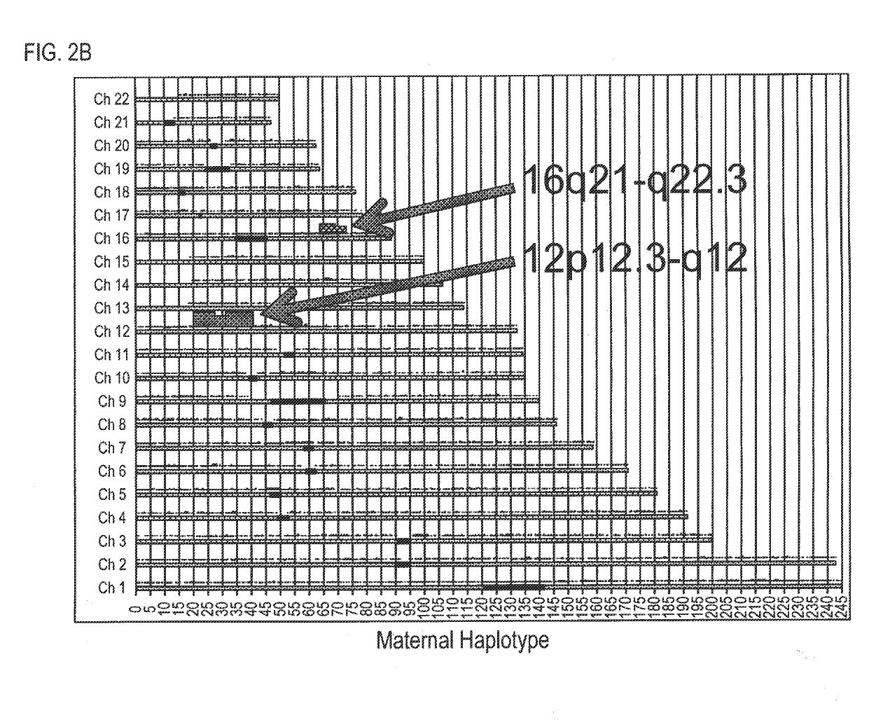

As shown in FIG. 2B, the phased haplotype sharing analysis identified two regions, 12p12.3-q12 (21 Mb) and 16q21-q22.3 (9 Mb); both inherited from the same mother and were shared by all 7 affected individuals and putative disease carriers in K1601. With reference to FIGS. 2A and 2B, the Y axis indicates the number of consecutive inclusion markers in each shared haplotype block, and the X axis indicates chromosomal position.

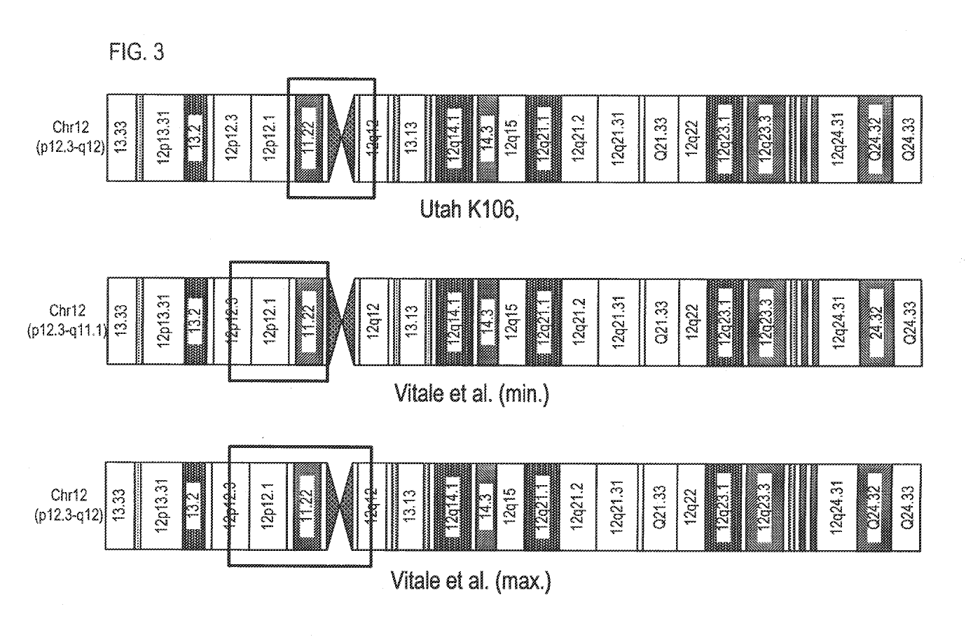

As shown in FIG. 3, the identified chromosome 12 MS region from the Utah 1601 family overlaps with the MS region in another multiplex MS family described by Vitale et al. (Vitale et al., Human Molecular Genetics, 2002, Vol. 11, No. 3, 295-300).

### Example 3

Linkage analysis was carried out on the 19 multigenerational Utah families with MS, including K1601, using the same Affymetrix Genome-Wide Human SNP array 6.0 genotype data. Accumulative hLOD scores of 6.4 and 4.5 were obtained for 12p12.3-q12 and 16q21-q22.3, respectively. 11 multigenerational MS families, including K1601, supported chromosome 12 and 16 linkages.

### Example 4

A custom targeted-enrichment system (Agilent SureSelect) was designed to selectively capture promoter and exonic regions from genes within the shared regions 12p12.3-q12 (95 genes) and 16q21-q22.3 (152 genes). Nucleotide sequence data was obtained from an Illumina HiSeq instrument (Illumina Inc, San Diego, Calif.).

Promoter and exonic regions were captured, sequenced, and analyzed from samples from 25 family members affected with MS, from 11 families that supported chromosome 12 and 16 linkages, including six members from K1601. Reference sequence assembly of 50 by paired-end reads was completed using the Burrows Wheeler Alignment method (BWA, Li H & Durbin R, 2010) and NovoAlign (novocraft.com).

Nucleotide sequence variants were annotated with ANNOVAR (Wang K, Li M, & Hakonarson H, 2010) and the DNA sequence analysis module in the SNP & Variation Suite software (Golden Helix, Inc., Bozeman, Mont.). Variant prioritization was carried out with the VAAST (Variant Annotation, Analysis & Search Tool) program (Yandell M, et al., 2011), a probabilistic search tool that identifies damaged genes and candidate disease causing variants in personal genome sequences. The 1000 Genome Project data was used as the background genome for the VAAST analysis.

Each putative candidate disease causing variant detected by in silico analysis was validated by visually inspecting the variant call quality on the Integrative Genome Viewer (IGV, BroadInstitute, Cambridge, Mass.). Variants that passed visual inspection were then subject to the LightScanner High resolution Melting analysis (Biofire Diagnostics, Inc., Salt Lake City, Utah) for DNA sequence variant detection. Any samples that gave abnormal melting profiles were sequenced by conventional Sanger method for confirmation. Validated functionally relevant MS candidate variants from chromosome 16 are listed in Table 1. The forward and reverse PCR primers designed to assay for the identified variants in Table 1 are also listed.

TABLE 1

| VAAST | | | | Chromosome | | | | A.A. | | | | |
|---|---|---|---|---|---|---|---|---|---|---|---|---|
| p- | VAAST | | Gene | Position | Ref. | Variant | RefSeq | Po- | Ref. | Var. | Forward | Reverse |
| value | score | Gene | Description | (hg19) | Allele | Allele | Transcript | sition | A.A. | A.A. | Primer | Primer |
| 4.60E- | 40.83 | ELMO3 | engulfment | chr16: | T | A | NM_024712 | 497 | C | S | ACTCCA | CACCTTG |
| 18 | | | and cell | 67236368 | | | | | | | GGCTCT | TCGAAGT |
| | | | motility 3 | | | | | | | | GAGACA | CCTCCT |
| | | | | | | | | | | | GC | (SEQ ID |
| | | | | | | | | | | | (SEQ ID | NO: 2) |
| | | | | | | | | | | | NO: 1) | |
| 4.60E- | 40.63 | ZFHX3 | zinc finger | chr16: | A | C | NM_006885 | 186 | C | G | TATTCG | CCTCGCT |
| 18 | | (ATBF1) | homeobox 3 | 72993489 | | | | | | | GGAAAG | TTTCCTG |
| | | | (ataxia | | | | | | | | CCTGGT | AACTCT |
| | | | telangi- | | | | | | | | CT | (SEQ ID |
| | | | ectasia | | | | | | | | (SEQ ID | NO: 4) |
| | | | motif | | | | | | | | NO: 3) | |
| | | | binding | | | | | | | | | |
| | | | factor 1) | | | | | | | | | |
| 4.60E- | 34.03 | IL34 | interleu- | chr16: | T | C | NM_001172772, | 57 | Y | H | GGAGCC | CAGGAAG |
| 18 | | | kin 34 | 70690511 | | | NM_001172771, | | | | TGCTGG | GGATTCT |
| | | | | | | | NM_152456 | | | | TCATTT | CACCAG |
| | | | | | | | | | | | CT | (SEQ ID |
| | | | | | | | | | | | (SEQ ID | NO: 6) |
| | | | | | | | | | | | NO: 5) | |

With further reference to Table 1, for the variant detected within the gene ELMO3, the reference allele is T and the variant (disease) allele is A, based on the RefSeq NM_{—}024172, resulting in a cysteine to serine change at amino acid 497 (C497S). ELMO3 has been associated with embryonic CNS development in Drosophila (Biersmith B, et al., PLoS One. 2011 Jan. 25; 6(1):e16120). For the variant detected within the gene ZFHX3, the reference allele is A and the variant (disease) allele is C, based on the RefSeq NM_{—}006885, resulting in a cysteine to glycine change at amino acid 186 (C186G). ZFHX3 (ATBF1) has been reported as being involved in neuronal differentiation and in protection of cerebellar neurons from oxidative stress (Jung C G, et al., Development. 2005 December; 132(23):5137-45. Epub 2005 Oct. 26; Kim T S, et al., Dis Model Mech. 2010 November-December; 3(11-12):752-62). For the variant detected within the gene IL34 (rs118062333), the reference allele is T and the variant (disease) allele is C (ESP 6500 all: C=29, T=12,967), based on the RefSeq NM_{—}001172772, NM_{—}001172771, and NM_{—}152456, resulting in a tyrosine to histidine change at amino acid 57 (Y57H). An inhibitor of IL34 has been used to treat MS in a pre-clinical trial setting (Five Prime Therapeutics, Inc., fiveprime.com/pipeline/fpa008).

Additional validated functionally relevant MS candidate variants from chromosomes 1, 2, 3, 6, 7, 10, 11, 12, 13, 14, 16, 18, and 19 are listed in Table 2.

TABLE 2

| | | Chromosome | | | | |
|---|---|---|---|---|---|---|
| | Gene | Position | Ref. | Variant | Forward | Reverse |
| Gene | Description | (hg19) | Allele | Allele | Primer | Primer |
| C1orf125 | hypothetical | chr1:179352648 | T | A | GCCATTTGAGAATTCC | GGCTTTGCAATTAC |
| | protein | | | | CTGT | CTTCGT |
| | LOC126859 | | | | (SEQ ID NO: 7) | (SEQ ID NO: 8) |
| PLD5 | GC phospholipase | chr1:242383291 | T | C | GCGTGCTTTATGAAG | TTCTGGATCGTGGA |
| | D5 | | | | GTGGT | CAAACA |
| | | | | | (SEQ ID NO: 9) | (SEQ ID NO: 10) |
| NCKAP5 | Nck-associated | chr2:133489544 | G | A | GAGGAAGGCACTTCA | ACTCCCAGACTCGG |
| | protein 5 | | | | CGTTC | GAAATC |
| | | | | | (SEQ ID NO: 11) | (SEQ ID NO: 12) |
| NCKAP5 | Nck-associated | chr2:133971335 | C | T | CGGCAGTCAGGACTT | CCATGCCAGTGTCA |
| | protein 5 | | | | ACCAT | CCTCTA |
| | | | | | (SEQ ID NO: 13) | (SEQ ID NO: 14) |
| SCN9A | | chr2:167138320 | — | A | AGGTTGGGATCATTC | AACCCAGCAATCTA |
| | | | | | AGCAT | GGCTCT |
| | | | | | (SEQ ID NO: 15) | (SEQ ID NO: 16) |
| TUBA4A | | chr2:220115579 | G | A | AAAGCAGGCATTGGT | AGTTCCAGACCAAC |
| | | | | | GATCT | CTGGTG |
| | | | | | (SEQ ID NO: 17) | (SEQ ID NO: 18) |
| ZNF717 | zinc finger | chr3:75786681 | G | A | GTTTCTCCCCCGTGT | ATCGCAAGTCATTC |
| | protein 717 | | | | GAATA | CTCACC |
| | | | | | (SEQ ID NO: 19) | (SEQ ID NO: 20) |
| NPHP3 | | chr3:132407671 | C | T | CTGAGATTTCCAACG | CCCCAGGCCATAGT |
| | | | | | CCTGT | ACCTTT |
| | | | | | (SEQ ID NO: 21) | (SEQ ID NO: 22) |
| LEKR1 | leucine, | chr3:156697055 | — | T | AAAACTTCAGAAGGC | CACCAGTAAATCAC |
| | glutamate and | | | | GGTGA | TGCCAAAA |
| | lysine rich 1 | | | | (SEQ ID NO: 23) | (SEQ ID NO: 24) |
| EHHADH | | chr3:184911180 | C | T | ACACCACCAAGCTCC | CCGAGGCATTGTCA |
| | | | | | TTCAC | TTTCTT |
| | | | | | (SEQ ID NO: 25) | (SEQ ID NO: 26) |
| AIF1 | allograft | chr6:31584216 | G | A | ATGTCCCTGAAACGA | ATCTCTTGCCCAGC |
| | inflammatory | | | | ATGCT | ATCATC |
| | factor 1 | | | | (SEQ ID NO: 27) | (SEQ ID NO: 28) |
| HIVEP2 | human | chr6:143094843 | T | C | ACTGTGTGCGAATCAT | CATTGGGAGGTCCA |
| | immunodeficienty | | | | CAGC | ATGAAG |
| | virus type I | | | | (SEQ ID NO: 29) | (SEQ ID NO: 30) |
| | enhancer | | | | | |
| RELN | reelin | chr7:103163905 | C | T | TGGCCACTGCACATG | CTTTTCCACAGCATC |
| | | | | | TCTAT | CTTCA |
| | | | | | (SEQ ID NO: 31) | (SEQ ID NO: 32) |
| RELN | reelin | chr7:103194141 | C | T | GGAAAACAACCAGGA | TTTTGGGCCCAGAG |
| | | | | | ATCCA | AAGAC |
| | | | | | (SEQ ID NO: 33) | (SEQ ID NO: 34) |
| IL2RA | | chr10:6063597 | C | A | CATTTTGCAGACGCTC | AACCTCCACCATGG |
| | | | | | TCAG | GAAAAT |
| | | | | | (SEQ ID NO: 35) | (SEQ ID NO: 36) |
| CD6ns | | chr11:60775079 | G | C | GTCTGACAAACGGGA | CCCAGTGCTCGGCA |
| | | | | | GCAG | CAC |
| | | | | | (SEQ ID NO: 37) | (SEQ ID NO: 38) |
| RAB38 | RAB38 | chr11:87847209 | G | T | ACACCAGCAAAGGTG | CTCCAGATGCCTGG |
| | | | | | CCTAC | TGAAAC |
| | | | | | (SEQ ID NO: 39) | (SEQ ID NO: 40) |
| PTPRO | receptor-type | chr12:15654574 | A | G | TCTGCTTGTGTACCTG | CTGGGAAATTGCCA |
| | protein tyrosine | | | | ACTCATT | CTCTGT |
| | phosphatase O | | | | (SEQ ID NO: 41) | (SEQ ID NO: 42) |
| PTPRO | receptor-type | chr12:15702050 | — | T | TTTCAATGCTATTCTT | AAGAAGGCTGGAGA |
| | protein tyrosine | | | | TGTCATCTT | TGGTCA |
| | phosphatase O | | | | (SEQ ID NO: 43) | (SEQ ID NO: 44) |
| STRAP | serine/threonine | chr12:16035712 | G | A | AAGACAACGACGACC | TGCAAGCGCTGATT |
| | kinase receptor | | | | CTCAG | AAGAAA |
| | associated | | | | (SEQ ID NO: 45) | (SEQ ID NO: 46) |
| PIK3C2G | phosphoinositide- | chr12:18719887 | C | T | TTGGCCCTTCCATCTG | AAAGGTAGGTGCCA |
| | 3-kinase, class 2 | | | | ATAC | CCAATG |
| | gamma | | | | (SEQ ID NO: 47) | (SEQ ID NO: 48) |
| PLEKHA5 | | chr12:19500111 | C | T | CAGAGCAGCCTCCCA | TCAAAAATGAGAGG |
| | | | | | TAATC | ACATGTAGGA |
| | | | | | (SEQ ID NO: 49) | (SEQ ID NO: 50) |
| PDE3A | | chr12:20522514 | A | C | GCTGGGGAGACCTGG | GAGTAAGTGATCCT |
| | | | | | TG | CCCCGAC |
| | | | | | (SEQ ID NO: 51) | (SEQ ID NO: 52) |
| GYS2 | glycogen | chr12:21690035 | C | G | CCTTTCAGCCTCCTCT | TGTCTTTGGCAGAC |
| | synthase 2 | | | | TCCT | AGAAGG |
| | | | | | (SEQ ID NO: 53) | (SEQ ID NO: 54) |
| ERGIC2 | PTX1 protein | chr12:29498389 | G | A | CGGTTGGATTCAACTT | GGCAGTAGTTTTGT |
| | | | | | ACCC | TGCTACTGA |
| | | | | | (SEQ ID NO: 55) | (SEQ ID NO: 56) |
| ABCD2 | ATP-binding | chr12:40013392 | G | C | GGGATAGAGGGTTTT | CATTTGCTGGGGAT |
| | cassette, sub- | | | | CAGAGC | TTCTGT |
| | family D, member | | | | (SEQ ID NO: 57) | (SEQ ID NO: 58) |
| | 2 | | | | | |
| COL2A1 | | chr12:48381394 | G | A | CCCACAACTGTCAGA | TCCTGAAGGTGCTC |
| | | | | | GCAAA | AAGGTC |
| | | | | | (SEQ ID NO: 59) | (SEQ ID NO: 60) |
| OR10AD1 | olfactory | chr12:48596875 | — | A | GGAGACAATGTGGTC | CATGAATGGCCTCA |
| | receptor, family | | | | CCTGA | TCATCTT |
| | 10, subfamily AD | | | | (SEQ ID NO: 61) | (SEQ ID NO: 62) |
| FMNL3 | | chr12:50043661 | C | T | GTTCCACCTTGCTGAA | CCACAGGTTTGACT |
| | | | | | GAGC | TGCAGA |
| | | | | | (SEQ ID NO: 63) | (SEQ ID NO: 64) |
| SLC11A2 | solute carrier | chr12:51386017 | G | T | CTTGGTACCCAAGGG | CTGCTTGTTGCTGT |
| | family 11 | | | | CAGTA | CTTCCA |
| | | | | | (SEQ ID NO: 65) | (SEQ ID NO: 66) |
| KRT80 | keratin 80 | chr12:52585500 | C | T | ATTGAGGGCCTTCAT | GCTGGCACTATCTC |
| | | | | | CTCCT | CAAGGT |
| | | | | | (SEQ ID NO: 67) | (SEQ ID NO: 68) |
| KRT75 | keratin 75 | chr12:52822050 | C | T | AGCCCAATTCTGAACT | ATCAAGCTGGCCCT |
| | | | | | GCAT | GGAC |
| | | | | | (SEQ ID NO: 69) | (SEQ ID NO: 70) |
| KRT74 | keratin 6 irs4 | chr12:52962049 | C | T | TAGGTGGCAATCTCC | AGACAATGCCCTGA |
| | | | | | ATGTC | AGGATG |
| | | | | | (SEQ ID NO: 71) | (SEQ ID NO: 72) |
| KRT74 | keratin 6 irs4 | chr12:52962050 | G | A | TAGGTGGCAATCTCC | AGACAATGCCCTGA |
| | | | | | ATGTC | AGGATG |
| | | | | | (SEQ ID NO: 73) | (SEQ ID NO: 74) |
| KRT76 | keratin 76 | chr12:53170712 | A | C | CTGAATTCCCCCAGG | TGGCAGAGGAGTAG |
| | | | | | AAAG | GTAGTGG |
| | | | | | (SEQ ID NO: 75) | (SEQ ID NO: 76) |
| KRT3 | keratin 3 | chr12:53184620 | — | A | CTGGATTAGTGCAGA | ACTTTCTCTTGCTTT |
| | | | | | TATTTCAGA | CTCAAACAGG |
| | | | | | (SEQ ID NO: 77) | (SEQ ID NO: 78) |
| ITGB7 | integrin, beta 7 | chr12:53590580 | G | A | GGAGGGTACTGTGCT | CTCTCCCATCACCG |
| | precursor | | | | CACAAA | TCCTC |
| | | | | | (SEQ ID NO: 79) | (SEQ ID NO: 80) |
| UTP20 | down-regulated | chr12:101763659 | A | G | AGTGCCTCATCTGGG | TGAAGACATCTCAC |
| | in metastasis | | | | TCTTG | CTTGAAACA |
| | | | | | (SEQ ID NO: 81) | (SEQ ID NO: 82) |
| TUBA3C | tubulin, alpha 3c | chr13:19751579 | C | T | GACGCTCGATGTCCA | GCAAGAAGTCCAAG |
| | | | | | GGTT | CTAGAAT |
| | | | | | (SEQ ID NO: 83) | (SEQ ID NO: 84) |
| SLITRK6 | slit and trk | chr13:86368978 | C | G | TGCAGGAGTGGTGAC | TGACATCCTCTGCA |
| | like 6 | | | | CATAA | CTTCC |
| | | | | | (SEQ ID NO: 85) | (SEQ ID NO: 86) |
| NUBPL | nucleotide | chr14:32142689 | G | T | TGGTATTGCTTGGTGA | CAATGGCCGACATT |
| | binding protein- | | | | GCAT | ACCATA |
| | like | | | | (SEQ ID NO: 87) | (SEQ ID NO: 88) |
| SNX29 | sorting nexin 29 | chr16:12662448 | G | A | TGCTCTTTCAGCGACA | TTTGTCGAGGTCAG |
| | | | | | TCAC | AGGTCA |
| | | | | | (SEQ ID NO: 89) | (SEQ ID NO: 90) |
| CNOT1 | | chr16:58616973 | C | T | CATGTGTTCACTTCTG | CAAGGGTTTTGGGA |
| | | | | | GTCGAT | ATGATG |
| | | | | | (SEQ ID NO: 91) | (SEQ ID NO: 92) |
| GOT2 | | chr16:58752137 | C | T | ATCGTCCTCACCTTCA | TCCTTTAAAGCTGCA |
| | | | | | CCAC | AAGTCG |
| | | | | | (SEQ ID NO: 93) | (SEQ ID NO: 94) |
| CDH11 | cadherin 11 | chr16:65038717 | T | G | CTCCTTGCCCTTCTCA | TGTGCCTACCACGT |
| | | | | | TGGT | AACCAA |
| | | | | | (SEQ ID NO: 95) | (SEQ ID NO: 96) |
| CDH16 | cadherin 16 | chr16:66946243 | G | A | AGGCCAAAAGTCCCT | GGCCTATAAGCCTC |
| | | | | | TCTGT | CCTGAG |
| | | | | | (SEQ ID NO: 97) | (SEQ ID NO: 98) |
| C16orf70 | lin-10 | chr16:67144115 | G | A | AATGCTGGACCTGGA | GGGCGTGAGAGACT |
| | | | | | GGTAG | GAGAAC |
| | | | | | (SEQ ID NO: 99) | (SEQ ID NO: 100) |
| FAM65A | hypothetical | chr16:67572934 | A | G | TGGAGAGCCGAGTTC | TCCACACCAAAGAG |
| | protein | | | | ATTCT | GCAAAG |
| | LOC79567 | | | | (SEQ ID NO: 101) | (SEQ ID NO: 102) |
| RLTPR | RGD motif, | chr16:67683449 | A | G | GGCTGAGTCTCGGCT | ACCGGAGCCTCGAG |
| | leucine rich | | | | GAA | TTGAC |
| | repeats, | | | | (SEQ ID NO: 103) | (SEQ ID NO: 104) |
| | tropomodulin | | | | | |
| PARD6A | par-6 | chr16:67696498 | G | A | CAGTTCCAATGGGCT | AGAGGCTGAAGCCA |
| | partitioning | | | | GTCTC | CTACCA |
| | defective 6 | | | | (SEQ ID NO: 105) | (SEQ ID NO: 106) |
| | homolog alpha | | | | | |
| C16orf48 | hypothetical | chr16:67697180 | C | T | ACTTTGGGCCGAGAA | CTGCTGCGTGAGCT |
| | protein | | | | AAGAT | GGTACT |
| | LOC84080 | | | | (SEQ ID NO: 107) | (SEQ ID NO: 108) |
| TSNAXIP | translin- | chr16:67859098 | A | G | AGCTGCATACAGGGG | TCATTCAGGTCTGC |
| 1 | associated | | | | TCCAG | GATGAG (SEQ ID |
| | factor | | | | (SEQ ID NO: 109) | NO: 110) |
| | X interacting | | | | | |
| | protein 1 | | | | | |
| TSNAXIP | | chr16:67859581 | G | — | CTGAGAAAGCCACGG | CCAAAGTTGGCCTT |
| 1 | | | | | TGACA | CATGTT |
| | | | | | (SEQ ID NO: 111) | (SEQ ID NO: 112) |
| SLC12A4 | solute carrier | chr16:67984868 | C | T | GAGTGTGTGGGGTGT | TGACGCCCAGAAGT |
| | family 12, | | | | CTGTG | CTATCC |
| | member 4 | | | | (SEQ ID NO: 113) | (SEQ ID NO: 114) |
| COG8 | | chr16:69373451 | G | A | GCTACCGATGGGATA | CGGAAATGTGCCTG |
| | | | | | GTCG | TTTCTT |
| | | | | | (SEQ ID NO: 115) | (SEQ ID NO: 116) |
| FUK | fucokinase | chr16:70508729 | T | G | ATAGGGGGCTGGAGT | CATGAGGCTGGCAG |
| | | | | | GACA | TAGTCC |
| | | | | | (SEQ ID NO: 117) | (SEQ ID NO: 118) |
| HYDIN | hydrocephalus | chr16:70852283 | C | T | AGTTGGCAGGCACCA | TTTCAGGGTCATCC |
| | inducing | | | | CAA | CTTCAG |
| | | | | | (SEQ ID NO: 119) | (SEQ ID NO: 120) |
| HYDIN | hydrocephalus | chr16:70867982 | C | A | CAGGAGCCCCATGCA | GCCCTTCCACAACA |
| | inducing | | | | TTC | TCACAC |
| | | | | | (SEQ ID NO: 121) | (SEQ ID NO: 122) |
| HYDIN | | chr16:70908206 | A | C | ACCCAGTAGCCAACA | TGAGGATGACATCA |
| | | | | | CTTGC | CCTTGG |
| | | | | | (SEQ ID NO: 123) | (SEQ ID NO: 124) |
| HYDIN | hydrocephalus | chr16:71015329 | G | T | GGGAGATCGAGGCAG | CGCGTAGGGCTTTA |
| | inducing | | | | ATTT | TCAGTT |
| | | | | | (SEQ ID NO: 125) | (SEQ ID NO: 126) |
| MARVEL | MARVEL domain | chr16:71674405 | T | A | TGGTCTTGAATGGGA | GAGCGGAGGATCGT |
| D3 | containing 3 | | | | TGGTT | GTACTG |
| | | | | | (SEQ ID NO: 127) | (SEQ ID NO: 128) |
| PHLPP2 | PH domain and | chr16:71686754 | C | T | TGGGCCTTCATCACC | AGCCAGTGCCCCTC |
| | leucine rich | | | | TCTAC | TCTAA |
| | repeat protein | | | | (SEQ ID NO: 129) | (SEQ ID NO: 130) |
| PKD1L3 | polycystin | chr16:71981414 | — | TTTG | AGTTACCCAAGAGTG | TGCCCACCTCTTCC |
| | 1-like | | | | CCAAGA | TTTACA |
| | 3 precursor | | | | (SEQ ID NO: 131) | (SEQ ID NO: 132) |
| ZFHX3 | zinc finger | chr16:72828144 | A | G | GGAACAGTCATCGTT | CATGGAATTGTCAC |
| (ATBF1) | homeobox 3 | | | | GTCCA | CCAGAA |
| | | | | | (SEQ ID NO: 133) | (SEQ ID NO: 134) |
| MLKL | mixed lineage | chr16:74712823 | G | C | AGCACCAGTCATGCA | CCTTTGGCTGTGTC |
| | kinase domain- | | | | GGTTT | AGGCTA |
| | like | | | | (SEQ ID NO: 135) | (SEQ ID NO: 136) |
| FA2H | | chr16:74750266 | G | — | CACCTGACTTCTGATG | CCCATTACTACCTG |
| | | | | | TGCAA | CACTTTGG |
| | | | | | (SEQ ID NO: 137) | (SEQ ID NO: 138) |
| WDR59 | | chr16:74983696 | G | A | AGGTGTCCACAGAGC | TCGATGTGCTAGTG |
| | | | | | TGGTAA | CTTTGG |
| | | | | | (SEQ ID NO: 139) | (SEQ ID NO: 140) |
| ZNRF1 | zinc and ring | chr16:75033679 | A | T | GGGTCTCCACCGATG | GGGGGTGTAGAGG |
| | finger protein | | | | ACA | CCAAAG |
| | 1 | | | | (SEQ ID NO: 141) | (SEQ ID NO: 142) |
| BCAR1 | breast cancer | chr16:75269325 | C | T | CGGCTCCTGTGGCTC | CTGGAGCTGGAAGT |
| | anti-estrogen | | | | AGA | TGCTG |
| | resistance 1 | | | | (SEQ ID NO: 143) | (SEQ ID NO: 144) |
| ADAT1 | adenosine | chr16:75654685 | C | T | GCCTTGTCAGCTGGA | ACCAGCTCAGACCA |
| | deaminase, | | | | GATTG | TGTGGA |
| | tRNA-specific 1 | | | | (SEQ ID NO: 145) | (SEQ ID NO: 146) |
| KARS | lysyl-tRNA | chr16:75665690 | T | C | CCATCTTCTCCGTGAT | CGACCGGGTTTATG |
| | synthetase | | | | TTCC | AAATTG |
| | | | | | (SEQ ID NO: 147) | (SEQ ID NO: 148) |
| KIAA1012 | hypothetical | chr18:29447411 | G | A | AAAAATTTGTTTTCAC | CAAGTGAACCTGAA |
| | protein | | | | CTTACTTTCA | ATGATTGG |
| | LOC22878 | | | | (SEQ ID NO: 149) | (SEQ ID NO: 150) |
| CPAMD8 | | chr19:17056375 | C | T | CAGCTTCATGGCTGA | ACACCCTGAGGAGA |
| | | | | | AGGA | ATCACG |
| | | | | | (SEQ ID NO: 151) | (SEQ ID NO: 152) |
| CPAMD8 | C3 and PZP-like, | chr19:17108094 | C | T | ATGGCCCAGATGCTG | CCCTCAAAGACACT |
| | alpha-2- | | | | ACC | CCAACC |
| | macroglobulin | | | | (SEQ ID NO: 153) | (SEQ ID NO: 154) |
| | domain | | | | | |
| CPAMD8 | C3 and PZP-Iike, | chr19:17108127 | C | T | GGGATCATGTCCCTC | CCTCCAAGCAGCTG |
| | alpha-2- | | | | ACG | AAGAGT |
| | macroglobulin | | | | (SEQ ID NO: 155) | (SEQ ID NO: 156) |
| | domain | | | | | |

Those having skill in the art will appreciate that many changes may be made to the details of the above-described embodiments without departing from the underlying principles of the invention.

1. A method of determining the risk of multiple sclerosis (MS), the method comprising:
assaying a biological sample of a subject for the presence of at least one variant allele listed in Table 1 and Table 2;
wherein the presence of the at least one variant allele is indicative of a risk of MS.

2. The method of claim 1, wherein the sample is assayed for the presence of at least one variant allele listed in Table 1 and Table 2 with at least one assay selected from a PCR assay, SNP array, PCR-based SNP genotyping, DNA hybridization, fluorescence microscopy, and immunoassay.

3. The method of claim 1, wherein the sample is assayed for the presence of at least one variant allele listed in Table 1 and Table 2 with a PCR assay using one or more of the PCR primer sequences selected from SEQ ID NOS: 1-156.

4. The method of claim 1, wherein the sample is assayed for the presence of at least one variant allele listed in Table 1 with a PCR assay using one or more of the PCR primer sequences selected from SEQ ID NOS: 1-6.

5. The method of claim 1, wherein the sample is assayed for the presence of at least one variant allele listed in Table 1 by assaying the sample for the presence of at least one of the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is A; the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is C; and the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is C.

6. The method of claim 5, wherein the sample is assayed for the presence of both the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is A; and the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is C.

7. The method of claim 5, wherein the sample is assayed for the presence of both the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is A; and the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is C.

8. The method of claim 5, wherein the sample is assayed for the presence of both the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is C; and the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is C.

9. The method of claim 1, wherein the sample is assayed for the presence of at least one variant allele listed in Table 2 with a PCR assay using one or more of the forward and reverse primers sequences selected from SEQ ID NOS: 7-156.

10. The method of claim 1, wherein the presence of the at least one variant allele indicates a diagnosis of MS in the subject.

11. An in vitro diagnostic product for detecting the risk of MS in a subject, the product comprising:
at least one laboratory test for assaying a biological sample of a subject for the presence of at least one variant allele listed in Table 1 and Table 2
wherein the presence of the at least one variant allele is indicative of a risk of MS.

12. The in vitro diagnostic product of claim 11, wherein the sample is assayed for the presence of at least one variant allele listed in Table 1 and Table 2 with at least one assay selected from a PCR assay, SNP array, PCR-based SNP genotyping, DNA hybridization, fluorescence microscopy, and immunoassay.

13. The in vitro diagnostic product of claim 11, wherein the sample is assayed for the presence of at least one variant allele listed in Table 1 and Table 2 with a PCR assay using one or more of the PCR primer sequences selected from SEQ ID NOS: 1-156.

14. The in vitro diagnostic product of claim 11, wherein the sample is assayed for the presence of at least one variant allele listed in Table 1 with a PCR assay using one or more of the PCR primer sequences selected from SEQ ID NOS: 1-6.

15. The in vitro diagnostic product of claim 11, wherein the sample is assayed for the presence of at least one variant allele listed in Table 1 by assaying the sample for the presence of at least one of the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is A; the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is C; and the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is C.

16. The in vitro diagnostic product of claim 15, wherein the sample is assayed for the presence of both the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is A; and the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is C.

17. The in vitro diagnostic product of claim 15, wherein the sample is assayed for the presence of both the chromosome 16 variant allele of Table 1, in the gene ELMO3, at position chr16:67236368, using the forward primer ACTCCAGGCTCTGAGACAGC (SEQ ID NO: 1) and the reverse primer CACCTTGTCGAAGTCCTCCT (SEQ ID NO: 2), wherein the variant allele is A; and the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is C.

18. The in vitro diagnostic product of claim 15, wherein the sample is assayed for the presence of both the chromosome 16 variant allele of Table 1, in the gene ZFHX3 (ATBF1), at position chr16:72993489, using the forward primer TATTCGGGAAAGCCTGGTCT (SEQ ID NO: 3) and the reverse primer CCTCGCTTTTCCTGAACTCT (SEQ ID NO: 4), wherein the variant allele is C; and the chromosome 16 variant allele of Table 1, in the gene IL34, at position chr16:70690511, using the forward primer GGAGCCTGCTGGTCATTTCT (SEQ ID NO: 5) and the reverse primer CAGGAAGGGATTCTCACCAG (SEQ ID NO: 6), wherein the variant allele is C.

19. The in vitro diagnostic product of claim 15, wherein the sample is assayed for the presence of at least one variant allele listed in Table 2 with a PCR assay using one or more of the forward and reverse primers sequences selected from SEQ ID NOS: 7-156.

20. The in vitro diagnostic product of claim 11, wherein the presence of the at least one variant allele indicates a diagnosis of MS in the subject.
